(19) United States
(12) Plant Patent Application Publication
(10) Pub. No.: US 2008/0189814 P1
(43) Pub. Date: Aug. 7, 2008

(54) Plant variety of photinia named 'Bindi Tip'

(76) Inventor: Thomas M. Foley JR., Woodland, CA (US)

Correspondence Address: CHRISTIE, PARKER & HALE, LLP, PO BOX 7068, PASADENA, CA, 91109-7068, US

(21) Appl. No.: 11/701,829

(22) Filed: Feb. 1, 2007

(51) Int. Cl. A01H 5/00

(52) U.S. Cl. PLT/263.1

(57) ABSTRACT

A new variety of Photina Rosaceae suitable for ornamental decoration, having a dense compact growth habit.

### CLASSIFICATION

The present invention relates to a new Photina plant.

### VARIETY DENOMINATION

The varietal denomination of the new plant is ‘Bindi Tip’.

### BACKGROUND OF THE INVENTION

The present invention relates to a new and distinct variety of Photinia which was discovered in an open pollinated seed block. The varietal denomination of the new plant is ‘Bindi Tip’.

The genus Photinia is included in the family Rosaceae which comprises about 3000 species of herbs, shrubs, trees and climbing plants growing in the northern and southern hemispheres. Photinia comprises over 60 species of trees and shrubs native to South and East Asia, many of which possess desirable ornamental characteristics.

### SUMMARY OF THE INVENTION

The new variety was discovered as a seeding of an unnamed, unpatented variety of Photinia. Asexual reproduction of the new variety by stem cuttings, performed in Vacaville, Calif. have confirmed that the distinctive characteristics of the new variety are stable and transmitted to succeeding generations, and the new variety reproduces true to type.

### COMPARISON WITH PARENT AND OTHER VARIETIES

‘Bindi Tip’ is distinguished from its parent and all other varieties of Photinia of which I am aware in having a more dense compact growth habit.

### BRIEF DESCRIPTION OF ILLUSTRATIONS

The accompanying photographic illustrations show a specimen of the new cultivar as true to color as is reasonably possible to make in an illustration of this character, wherein:

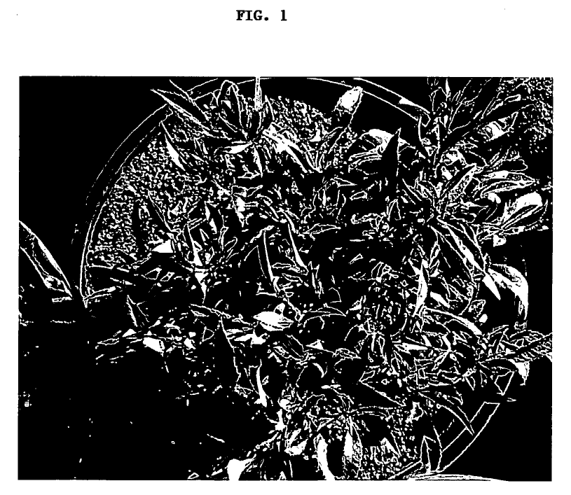

FIG. 1 shows a top view of an exemplary ‘Bindi Tip’ plant;

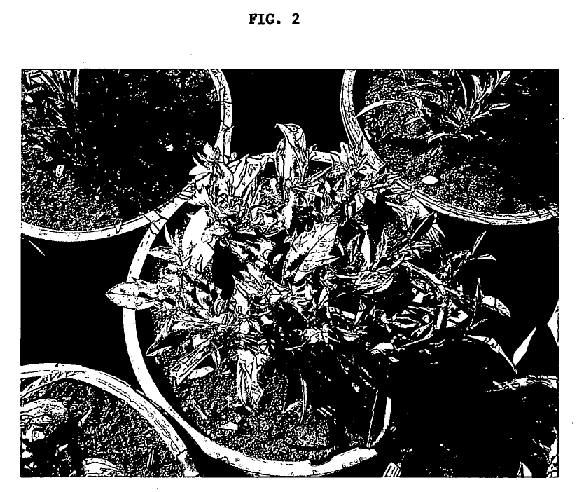

FIG. 2 shows second top view of an exemplary ‘Bindi Tip’ plant;

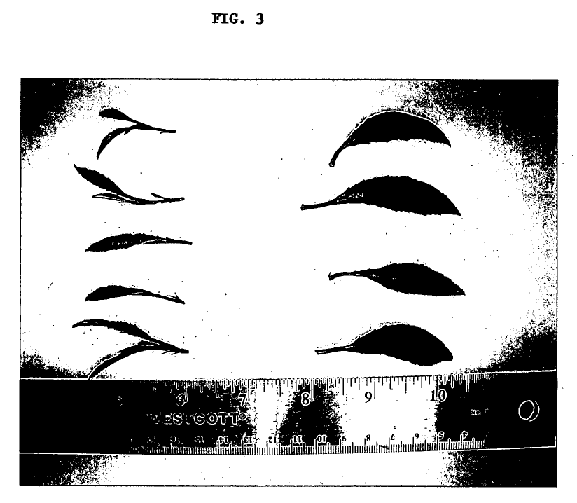

FIG. 3 shows a close-up of exemplary leaves from the ‘Bindi Tip’ plant shown in FIGS. 1 and 2;

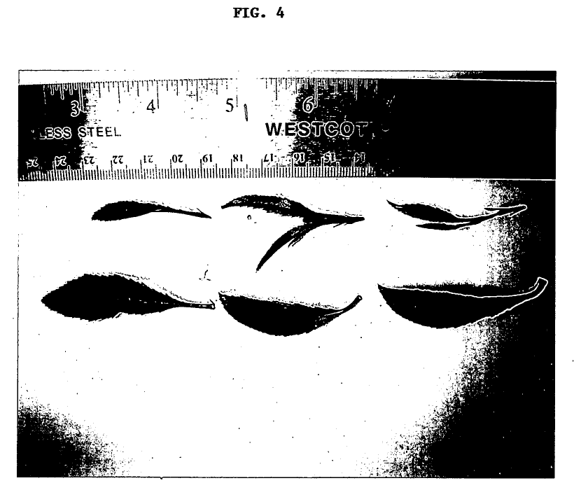

FIG. 4 shows a side-by side comparison of exemplary leaves from the ‘Bindi Tip’ plant shown in FIGS. 1 and 2; and

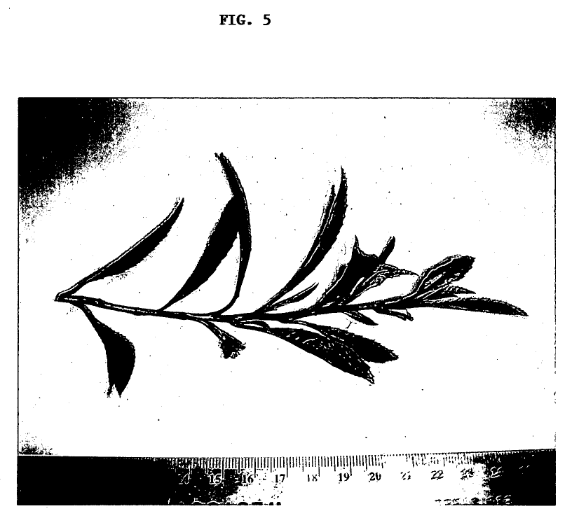

FIG. 5, shows a close-up of an exemplary branching of the ‘Bindi Tip’ plant shown in FIGS. 1 and 2.

### DETAILED DESCRIPTION OF THE NEW VARIETY

‘Bindi Tip’ has not been observed under all possible environmental, cultural and light conditions. The following observations and descriptions are of two-year-old plants grown in 5-gallon containers in Vacaville, Calif. In this description, color references are to The Royal Horticultural Society Colour Chart (2001) and terminology used in the color descriptions herein refers to plate numbers in this color chart. Phenotypic expression may vary with light intensity, cultural and environmental conditions.
Classification:
   Botanical.—Photinia×‘Bindi Tip’.
   Parentage.—Seeding selection from unknown parentage.
   Propagation.—By vegetative cuttings.
Plant:
   Size.—Mature plant can reach a height and spread to about 1 meter at maturity.
   Habit.—A dense dwarf broadleaf evergreen shrub.
   Branching.—Alternate, dense Size: Length: 20 cm to 45 cm Width: 1 mm to 4 mm Internodes: 6 to 10 mm.
Leaf:
   Arrangement.—Simple, alternate on stems.
   Leaf shape.—Lanceolate Apex: Acuminate to Acute Base: Atenuate.
   Leaf size.—Immature: From 20 to 35 mm long and 7 to 10 mm wide Mature: From 70 to 80 mm long and 15 to 20 mm wide.
   Leaf margin.—Serrate.
   Leaf venation.—Pinnate.
   Leaf texture and substance.—Adaxial: leathery, smooth Abaxial: leathery, smooth.
   Immature leaf color.—Adaxial: Near grey-brown group N199 A-B Abaxial: Near greyed-orange group 177 B Midrib: Near yellow-green group N144 A.
   Mature leaf color.—Adaxial: Near Green Group 139A Abaxial: Near Yellow-Green Group 146B.
   Mature stem color.—Near Grey-Brown Group 199 A-B.
   Immature stem color.—Near Greyed-Orange Group 175A.
Petioles:
   Immature.—Size: From 5 to 10 mm long Color: Near Greyed-Orange Group 175A.
   Mature.—Size: From 10 to 15 mm long Color: Near Yellow-Green Group 146B-C.
Other descriptors:
   Hardiness.—USDA Zone 7 (−20° F. to −10° F.).
   Vigor.—Slow, 10 cm per year.
   Pests/diseases.—No pest or disease problems noted.
Inflorescence: None Observed

What is claimed is:

1. A new plant variety of Photinia, the variety substantially as described and illustrated herein.
